(19) United States
(12) Patent Application Publication
(10) Pub. No.: US 2017/0342463 A1
(43) Pub. Date: Nov. 30, 2017

(54) DIGITAL PROXIMITY ASSAY

(71) Applicant: Bio-Rad Laboratories, Inc., Hercules, CA (US)

(72) Inventors: Vered BRONNER, Adi (IL); Dalia SHEZIFI, Kibutz Nahsholim (IL)

(21) Appl. No.: 15/602,667

(22) Filed: May 23, 2017

Related U.S. Application Data
(60) Provisional application No. 62/341,550, filed on May 25, 2016.

(51) Int. Cl. C12Q 1/68

(52) U.S. Cl. CPC C12Q 1/6804; C12Q 1/6818; C12Q 1/6834

(57) ABSTRACT

Methods of determining the presence or absence of a target in a sample are provided. Kits for performing the methods described herein are also provided.

This application claims the benefit of U.S. Application 62/341,550 filed on May 25, 2016 which is hereby incorporated by reference in its entirety.

### BACKGROUND

Proximity assays permit the sensitive, rapid and convenient detection or quantification of one or more analytes in a sample. Proximity assays utilize a pair of oligonucleotide-labeled antibodies to detect a specific target. The oligonucleotides are designed to hybridize, directly or through a linker, to form a new DNA template when the two antibodies are bound simultaneously to the target. The resulting DNA template is then detected using real-time polymerase chain reaction (PCR). To quantify the amount of target, a standard curve is used to correlate the Cq value to the DNA template and thereby the target concentration.

In proximity assays, the primary source of background signal is due to spontaneous binding of two oligonucleotide probes that are not bound to a target. To minimize background, proximity assays are performed at a relatively low probe concentration. Thus, the final number of DNA templates generated is lower than the actual number of target molecules. This can limit the sensitivity of the assay.

### SUMMARY

Disclosed herein are methods of detecting the presence or absence of a target in a sample.

In an embodiment, a method comprises contacting the sample with a first affinity agent linked to a solid support, wherein the first affinity agent specifically binds to a first epitope on the target; washing the solid support; incubating the solid support with a solution having a second affinity agent capable of specifically binding to a second epitope on the target and a third affinity agent capable of specifically binding to a third epitope on the target, wherein the second affinity agent is attached to a first oligonucleotide and the third affinity agent is attached to a second oligonucleotide capable of interacting directly or indirectly with the first oligonucleotide when the first and second oligonucleotides are in close proximity; allowing the first and second oligonucleotides to interact by (a) hybridization of a third oligonucleotide complementary to at least a portion of each of the first and second oligonucleotides followed by ligation of the first oligonucleotide to the second oligonucleotide; or (b)hybridization of the first oligonucleotide to the second oligonucleotide; washing the solid support; optionally forming a DNA template; releasing the DNA template and forming a solution comprising the DNA template; forming a plurality of partitions from the solution, wherein a subset of the partitions contains the DNA template; amplifying the DNA template in the subset of partitions; and detecting the presence or absence of the target in the sample by detecting the presence or absence of the amplified DNA template in the plurality of partitions. In some embodiments, the method further comprises quantifying the target by determining a number of partitions comprising the target and a total number of partitions.

In some embodiments, the first, second and third epitopes are at least partially overlapping. In some embodiments, the second epitope is located on a first target and the third epitope is located on a second target (e.g., a protein with non-identical subunits). In some embodiments, the target (e.g., a dimeric protein or an aggregate-forming protein) has a repeating identical epitope such that the first and second affinity agents or the first and third affinity agents recognize the same epitope.

In some embodiments, each of the plurality of partitions is a droplet. In some embodiments, the forming the DNA template step comprises extending the 3′ terminus of the third oligonucleotide. In certain embodiments, the forming the DNA template step comprises extending the 3′ terminus of the first oligonucleotide and/or the second oligonucleotide. In some embodiments, the hybridization of the first oligonucleotide to the second oligonucleotide comprises forming the DNA template step.

In some embodiments, the concentration of the second and third affinity agents is at least equal to the concentration of the first affinity agent. In certain embodiments, the releasing step comprises cleaving a site in the DNA template by a restriction endonuclease. In some embodiments, the releasing step comprises physical, chemical or enzymatic cleavage of the DNA template. In some embodiments, the releasing step comprises cleaving at least a portion of an affinity agent-oligonucleotide complex by a protease or by a change in pH.

In some embodiments, the amplifying step comprises PCR. In some embodiments, the target is a protein or a protein aggregate. In certain embodiments, the first, second, and third affinity agents are each an antibody or an antibody fragment. In some embodiments, the solid support is a particle or a surface of a reaction vessel.

In an embodiment, a kit for detecting the presence or absence of a target in a sample comprises a first affinity agent linked to a solid support, wherein the first affinity agent specifically binds to a first epitope on the target; and a second affinity agent capable of specifically binding to a second epitope on the target and a third affinity agent capable of specifically binding to a third epitope on the target, wherein the second affinity agent is conjugated to a first oligonucleotide and the third affinity agent is conjugated to a second oligonucleotide capable of interacting directly or indirectly with the first oligonucleotide when the first and second oligonucleotides are in close proximity. In some embodiments, the kit further comprises at least one component consisting of a ligase, a restriction endonuclease, a DNA polymerase, dNTPs, a buffer, a PCR master mix, and instructions for performing a method of detecting a target in a sample.

### BRIEF DESCRIPTION OF THE DRAWINGS

FIG. 1 is a flow chart showing a method of determining the presence or absence of a target according to an embodiment of the invention.

FIG. 2 depicts a scheme for determining the presence or absence of a target according to an embodiment of the invention.

### DETAILED DESCRIPTION

Described herein are methods for determining the presence or absence of a target in a sample. Digital proximity assay methods are described with which the concentration of a target can be determined without a standard curve. Digital proximity assay methods have also been discovered that have a reduced background of target-independent oligonucleotide probe binding.

### DEFINITIONS

Unless defined otherwise, technical and scientific terms used herein have the same meaning as commonly understood by a person of ordinary skill in the art. See, e.g., Lackie, DICTIONARY OF CELL AND MOLECULAR BIOLOGY, Elsevier (4th ed. 2007); Green et al., MOLECULAR CLONING, A LABORATORY MANUAL (FOURTH EDITION), Cold Spring Harbor Lab Press (Cold Spring Harbor, N.Y. 2012).

The term “a” or “an” is intended to mean “one or more.” The term “comprise,” and variations thereof such as “comprises” and “comprising,” when preceding the recitation of a step or an element, are intended to mean that the addition of further steps or elements is optional and not excluded. Any methods, devices and materials similar or equivalent to those described herein can be used in the practice of this invention. The following definitions are provided to facilitate understanding of certain terms used frequently herein and are not meant to limit the scope of the present disclosure.

As used herein, “nucleic acid” means a compound comprising a chain of nucleotide monomers. A nucleic acid can be single-stranded or double-stranded (i.e., base-paired with another nucleic acid), among others. The chain of a nucleic acid can be composed of any suitable number of monomers, such as at least about ten or one hundred, among others. Generally, the length of a nucleic acid chain corresponds to its source, with synthetic nucleic acids (e.g., nucleic acid reagents such as primers and probes) typically being shorter and biologically produced nucleic acids (e.g., nucleic acid analytes) typically being longer.

A nucleic acid can have a natural or artificial structure, or a combination thereof. Nucleic acids with a natural structure, namely, deoxyribonucleic acid (DNA) and ribonucleic acid (RNA), have a backbone of alternating pentose sugar groups and phosphate groups. Each pentose group is linked to a nucleobase (e.g., a purine (such as adenine (A) or guanine (T)) or a pyrimidine (such as cytosine (C), thymine (T), or uracil (U))). Nucleic acids with an artificial structure are analogs of natural nucleic acids and can, for example, be created by changes to the pentose and/or phosphate groups of the natural backbone. Exemplary artificial nucleic acids include glycol nucleic acids (GNA), peptide nucleic acids (PNA), locked nucleic acid (LNA), threose nucleic acids (TNA), and the like. Similarly, nucleic acids having an artificial structure are analogs of natural nucleic acids and can, for example, be created by changes to the nucleobase. Exemplary artificial or non-naturally occurring nucleobasee include, but are not limited to halogenated nucleobases (5-FU), hypoxanthine, xanthine, 7-methylguanine, inosine, xanthosine, 7-methyguanosine, 5,6-dihydrouracil, 5-methycytosine, 5-hydroxymethylcytosine, dihydrouridine, and 5-methylcytidine.

The sequence of a nucleic acid is defined by the order in which nucleobases are arranged along the backbone (typically read from the 5′ to 3′ end). This sequence generally determines the ability of the nucleic acid to bind specifically to a partner chain (or to form an intramolecular duplex) by hydrogen bonding. In particular, adenine pairs with thymine (or uracil) and guanine pairs with cytosine. A nucleic acid that can bind to another nucleic acid in an antiparallel fashion by forming a consecutive string of adenine-thymine and guanine-cytosine base pairs with the other nucleic acid is termed “complementary.”

The terms “label”, “detectable label”, or “labeling agent” refer to a composition detectable by spectroscopic, photochemical, biochemical, immunochemical, chemical, or other physical means. For example, useful labels include fluorescent dyes (fluorophores), fluorescent quenchers, luminescent agents, electron-dense reagents, enzymes (e.g., as commonly used in an ELISA), biotin, digoxigenin, ³²P and other isotopes, haptens, proteins, nucleic acids, or other substances which can be made detectable, e.g, by incorporating a label into an oligonucleotide, peptide, or antibody specifically reactive with a target molecule. The term includes combinations of single labeling agents, e.g., a combination of fluorophores that provides a unique detectable signature, e.g., at a particular wavelength or combination of wavelengths. A detectable label can also include a combination of a reporter and a quencher.

A molecule that is “linked” to a label (e.g., as for a labeled nucleic acid as described herein) is one that is bound, either covalently, through a linker or a chemical bond, or noncovalently, through ionic, van der Waals, electrostatic, or hydrogen bonds to a label such that the presence of the molecule can be detected by detecting the presence of the label bound to the molecule.

The term “reporter” refers to a substance or a portion thereof which is capable of exhibiting a detectable signal, which signal can be suppressed by a quencher. The detectable signal of the reporter is, e.g., fluorescence in the detectable range; thus, a reporter can also be a label.

The term “quencher” refers to a substance which is capable of suppressing, reducing, inhibiting, etc., the detectable signal produced by the reporter.

As used herein, the term “quenching” refers to a process whereby, when a reporter and a quencher are in close proximity, and the reporter is excited by an energy source, a substantial portion of the energy of the excited state non-radiatively transfers to the quencher where it either dissipates nonradiatively or is emitted at a different emission wavelength than that of the reporter (e.g., by fluorescence resonance energy transfer or FRET).

The reporter can be selected from fluorescent organic dyes modified with a suitable linking group for attachment to the oligonucleotide, such as to the 3′ or 5′ terminus. The quencher can also be selected from organic dyes, which may or may not be fluorescent, depending on the embodiment of the invention. Generally, whether the quencher is fluorescent or simply releases the transferred energy from the reporter by non-radiative decay, the absorption band of the quencher should at least substantially overlap the fluorescent emission band of the reporter to optimize the quenching.

Non-fluorescent quenchers or dark quenchers typically function by absorbing energy from excited reporters, but do not release the energy radiatively.

Selection of appropriate reporter-quencher pairs for particular probes can be undertaken in accordance with known techniques. Fluorescent and dark quenchers and their relevant optical properties from which exemplary reporter-quencher pairs can be selected are listed and described, for example, in R. W. Sabnis, HANDBOOK OF FLUORESCENT DYES AND PROBES, John Wiley and Sons, New Jersey, 2015, the content of which is incorporated herein by reference.

Reporter-quencher pairs can be selected from xanthene dyes including fluoresceins and rhodamine dyes. Many suitable forms of these compounds are available commercially with substituents on the phenyl groups, which can be used as the site for bonding or as the bonding functionality for attachment to an oligonucleotide. Another group of fluorescent compounds for use as reporters are the naphthylamines, having an amino group in the alpha or beta position. Included among such naphthylamino compounds are 1-dimethylaminonaphthyl-5 sulfonate, 1-anilino-8-naphthalene sulfonate and 2-p-touidinyl-6-naphthalene sulfonate. Other dyes include 3-phenyl-7-isocyanatocoumarin; acridines such as 9-isothiocyanatoacridine; N-(p-(2-benzoxazolyl)phenyl)maleimide; benzoxadiazoles; stilbenes; pyrenes and the like.

Suitable examples of quenchers can be selected from 6-carboxy-tetramethyl-rhodamine, 4-(4-dimethylaminophenylazo) benzoic acid (DABYL), tetramethylrhodamine (TAMRA), BHQ-O™, BHQ-1™, BHQ-2™, and BHQ-3™, each of which are available from Biosearch Technologies, Inc. of Novato, Calif., Qy7™ QSY-9™, QSY-21™ and QSY-35™, each of which are available from Molecular Probes, Inc.

Suitable examples of reporters can be selected from dyes such as SYBR green, 5-carboxyfluorescein (5-FAM™ available from Applied Biosystems of Foster City, Calif), 6-carboxyfluorescein (6-FAM), tetrachloro-6-carboxyfluorescein (TET), 2,7-dimethoxy-4,5-dichloro-6-carboxyfluorescein, hexachloro-6-carboxyfluorescein (HEX), 6-carboxy-2′,4,7,7′-tetrachlorofluorescein (6-TET™ available from Applied Biosystems), carboxy-X-rhodamine (ROX), 6-carboxy-4′,5′-dichloro-2′,7′-dimethoxyfluorescein (6-JOE™ available from Applied Biosystems), VIC™ dye products available from Molecular Probes, Inc., NED™ dye products available from Applied Biosystems, Cal Fluor dye products (such as, e.g., Cal Fluor Gold 540, Orange 560, Red 590, Red 610, Red 635) available from Biosearch Technologies, Quasar dye products (such as, e.g., Quasar 570, 670, 705) available from Biosearch Technologies, and the like.

“Target” or “target analyte” (used herein interchangeably) refers to any agent whose presence and/or amount is to be determined. In some embodiments, the target can be a mixture of several targets. In some embodiments the presence or concentration profile of the different targets is to be determined.

The target can be any biological and/or chemical agent (i.e., molecule/macromolecule/complex/conjugate). In some embodiments the target is an organic or inorganic molecule. In some embodiments the target is a biological agent.

In one embodiment the target is a protein, a protein aggregate, a polypeptide or a peptide. In some embodiments, the protein aggregate comprises more than one protein (e.g., more than one target). In some embodiments, the protein has more than one identical or non-identical subunit which may or may not be covalently bound to each other. The targets can be hormones, antibodies, amino acids (e.g., glutamic acid, aspartic acid) or any derivatives and/or combination thereof. In some embodiments the target is a toxin or a drug. In some embodiments, the amount of the target is in micrograms. In some embodiments, the amount of the target is below 1 microgram. In some embodiments, amount of the target is in nanograms. In some embodiments the amount of target is between 100 ng to 1 ng. In some embodiments, the amount of target is between 100 pg to 1 pg. In certain embodiments, the amount of target is between 1 pg to 1 fg.

The term “antibody” refers to a polypeptide of the immunoglobulin family or a polypeptide comprising fragments of an immunoglobulin that is capable of noncovalently, reversibly, and in a specific manner binding to a corresponding antigen. The term includes, but is not limited to, polyclonal or monoclonal antibodies of the isotype classes IgA, IgD, IgE, IgG, and IgM, derived from human or other mammalian cells, including natural or genetically modified forms such as humanized, human, single-chain, chimeric, synthetic, recombinant, hybrid, mutated, grafted, and in vitro generated antibodies. The term encompases conjugates, including but not limited to fusion proteins containing an immunoglobulin moiety (e.g., chimeric or bispecific antibodies or single chain Fv's (scFv's)), and fragments, such as Fab, F(ab′)2, Fv, scFv, Fd, dAb and other compositions.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively. The variable region contains the antigen-binding region of the antibody (or its functional equivalent) and is most critical in specificity and affinity of binding. See Paul, Fundamental Immunology (2003).

Antibodies can exist as intact immunoglobulins or as any of a number of well-characterized fragments that include specific antigen-binding activity. Such fragments can be produced by digestion with various peptidases. Pepsin digests an antibody below the disulfide linkages in the hinge region to produce F(ab)′₂, a dimer of Fab which itself is a light chain joined to V_{H}-C_{H}1 by a disulfide bond. The F(ab)′₂ can be reduced under mild conditions to break the disulfide linkage in the hinge region, thereby converting the F(ab)′₂ dimer into an Fab′ monomer. The Fab′ monomer is essentially Fab with part of the hinge region. While various antibody fragments are defined in terms of the digestion of an intact antibody, one of skill will appreciate that such fragments can be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., scFv) or those identified using phage display libraries (see, e.g., McCafferty et al. (1990) Nature 348:552-554). Methods for the preparation of antibodies are known in the art; see, e.g., Kohler & Milstein (1975) Nature 256:495-497; Kozbor et al. (1983) Immunology Today 4:72; Cole et al., Monoclonal Antibodies and Cancer Therapy, pp. 77-96. Alan R. Liss, Inc. 1985).

As used herein, the term “Fv” refers to a monovalent or bi-valent variable region fragment, and can encompass only the variable regions (e.g., V_{L} and/or V_{H}), as well as longer fragments, e.g., an Fab, Fab′ or F(ab′)2, which also includes C_{L} and/or C_{H}1. Unless otherwise specified, the term “Fc” refers to a heavy chain monomer or dimer comprising C_{H}1 and C_{H}2 regions.

The term “partitioning” or “partitioned” refers to separating an aqueous solution having one or more of a sample and reactant into a plurality of portions, or “partitions.” Partitions can be solid or fluid. In some embodiments, a partition is a solid partition, e.g., a microchannel or microwell. In some embodiments, a partition is a fluid partition, e.g., a droplet. In some embodiments, a fluid partition (e.g., a droplet) is a mixture of immiscible fluids (e.g., water and oil). In some embodiments, a fluid partition (e.g., a droplet) is an aqueous droplet that is surrounded by an immiscible carrier fluid (e.g., oil).

### Methods

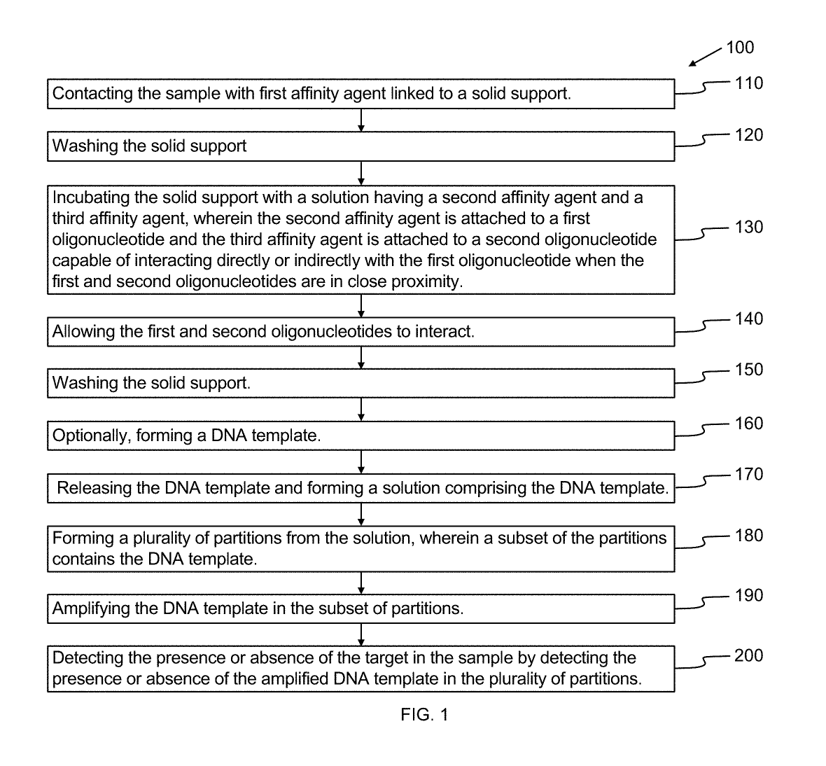

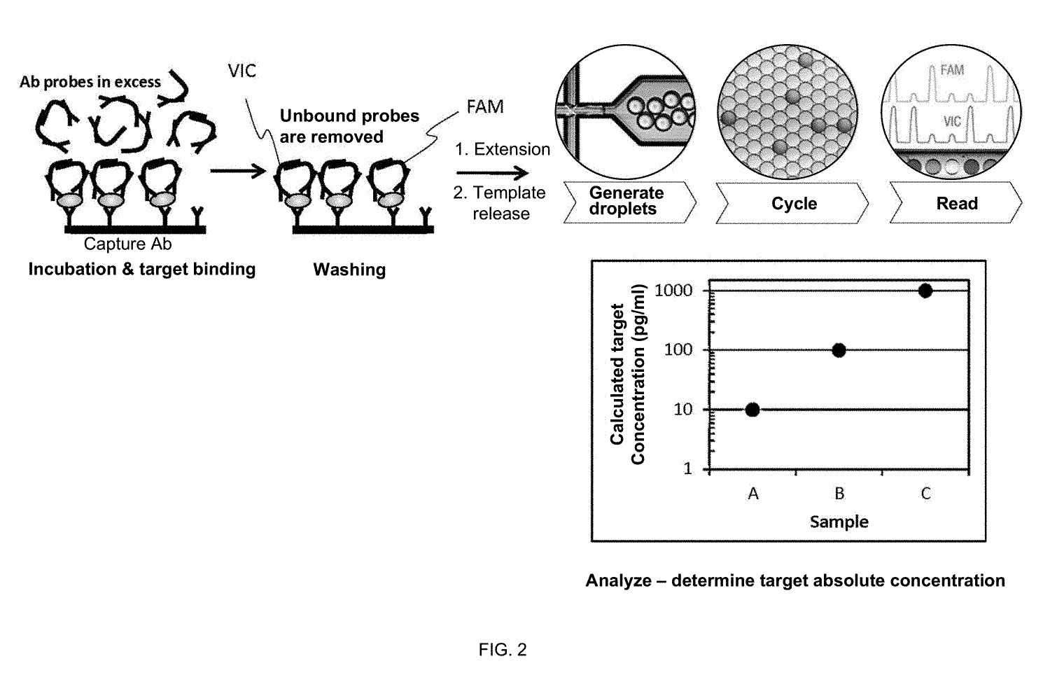

Referring to FIGS. 1 and 2, a method 100 of determining the presence or absence of a target in a sample will now be described. Some of the steps can be performed in any suitable order, in any suitable combination, and can be combined with or modified by any other suitable aspects of the disclosure provided herein.

In exemplary step 110, the sample is contacted with a first affinity agent (e.g., an antibody or antibody fragment) linked to a solid support, wherein the first affinity agent specifically binds to a first epitope on the target. In some embodiments, the sample is incubated with the first affinity agent to allow time for the first affinity agent to capture or bind to the target. In some embodiments, the sample is incubated with the first affinity agent for one hour or more. In some embodiments, prior to contacting the sample with the first affinity agent linked to the solid support, the solid support is treated with a blocking agent to prevent non-specific binding of material to the support. Exemplary blocking agents include, but are not limited to, proteins (e.g., non-fat milk or bovine serum albumin) and detergents (e.g., Tween 20 or Triton X-100).

In some embodiments, the sample is a biological sample. Biological samples can be obtained from any biological organism, e.g., an animal, plant, fungus, bacterial, or any other organism. In some embodiments, the biological sample is from an animal, e.g., a mammal (e.g., a human or a non-human primate, a cow, horse, pig, sheep, cat, dog, mouse, or rat), a bird (e.g., chicken), or a fish. A biological sample can be any tissue or bodily fluid obtained from the biological organism, e.g., blood, a blood fraction, or a blood product (e.g., serum, plasma, platelets, red blood cells, and the like), sputum or saliva, tissue (e.g., kidney, lung, liver, heart, brain, nervous tissue, thyroid, eye, skeletal muscle, cartilage, or bone tissue); cultured cells, e.g., primary cultures, explants, transformed cells, stem cells, stool, or urine. Other samples are non-biological and can include, but are not limited to, water and air.

In some embodiments, the sample can be prepared to improve the efficient detection of the target(s). For example, in some embodiments the sample can be fragmented, fractionated, homogenized, or sonicated. In some embodiments, a target of interest, or a sub-fraction comprising the target of interest, can be extracted or isolated from a sample (e.g., a biological sample). In some embodiments, the sample is enriched for the presence of the one or more targets. In some embodiments, the target is enriched in the sample by an affinity method, e.g., immunoaffinity enrichment. In some embodiments, the target is enriched in the sample using size selection (e.g., removing very small fragments or molecules or very long fragments or molecules).

Exemplary solid supports include, but are not limited to, particles (e.g., polymeric or magnetic beads) or the surface of reaction vessels (e.g., tubes or wells). In some embodiments, the solid support is not chemically modified prior to the attachment of the first affinity agent antibody (e.g., the antibody is attached to the substrate by non-covalent adsorption, based on hydrophobic and other interactions). In certain embodiments, the solid support is chemically modified prior to the attachment of the first affinity agent. Exemplary chemically modified solid supports can have carboxyl or amine groups attached and these groups can be used to covalently bind the first affinity agent. In some embodiments, the first affinity agent is attached to the solid support via carbodiimide mediated chemistry to form an amide bond. In some embodiments, the attachment of the first affinity agent to the solid support is enhanced by a chemical or a photochemical reaction. In certain embodiments, the first affinity agent is permanently attached to the solid support by a chemical or a photochemical reaction. In some embodiments, the solid support is deactivated after attaching the first affinity agent to the solid support to prevent binding of other agents. For example, active carboxyl groups on the solid support can be deactivated with ethanolamine.

In exemplary step 120, the solid substrate is washed with a wash solution (e.g., buffer having a detergent such as Tween 20 or Triton X-100) to remove unbound material.

In exemplary step 130, the solid support is incubated with a solution having a second affinity agent capable of specifically binding to a second epitope on the target and a third affinity agent capable of specifically binding to a third epitope on the target, wherein the second affinity agent is attached to a first oligonucleotide and the third affinity agent is attached to a second oligonucleotide capable of interacting directly or indirectly with the first oligonucleotide when the first and second oligonucleotides are in close proximity. In some embodiments, the first, second and third epitopes are at least partially overlapping. In some embodiments, the second epitope is located on a first target and the third epitope is located on a second target. In some embodiments, the target (e.g., a dimeric protein or an aggregate-forming protein) has a repeating identical epitope such that the first and second affinity agents or the first and third affinity agents recognize the same epitope.

In some embodiments, the second and third affinity agents are antibodies or antibody fragments. In certain embodiments, the concentration of the second and third affinity agents is at least equal to the concentration of the first affinity agent.

In exemplary step 140, the first and second oligonucleotides are allowed to interact by (a) hybridization of a third oligonucleotide complementary to at least a portion of each of the first and second oligonucleotides followed by ligation of the first oligonucleotide to the second oligonucleotide; or (b) hybridization of the first oligonucleotide to the second oligonucleotide.

In exemplary step 150, the solid substrate is again washed with a wash solution (e.g., buffer having a detergent such as Tween 20 or Triton X-100) to remove unbound material.

In exemplary step 160, a DNA template is formed. In some embodiments, the DNA template is formed by hybridizing the third oligonucleotide to the first and second oligonucleotides followed by ligating the first and second oligonucleotides. In some embodiments, the DNA template is formed by hybridizing the first oligonucleotide to the second oligonucleotide.

In exemplary step 170, the DNA template is released and a solution comprising the DNA template is formed. In some embodiments, the DNA template is released by cleaving a site in the DNA template by a restriction endonuclease. In certain embodiments, the DNA template is released by physical, chemical or enzymatic cleavage of the DNA template. In some embodiments, the DNA template is released by cleaving at least a portion of an affinity agent-oligonucleotide complex by a protease or by a change in pH.

In exemplary step 180, a plurality of partitions is formed from the solution such that a subset of the partitions contains the DNA template. The partitions can include any of a number of types of partitions, including solid partitions (e.g., wells or tubes) and fluid partitions (e.g., aqueous phase or droplet within an oil phase). In some embodiments, the partitions are droplets. In some embodiments, the partitions are microchannels or microwells. Methods and compositions for partitioning a solution are described, for example, in published patent applications WO 2012/135259, WO 2014/117088, WO 2010/036352, and U.S. Pat. No. 9,156,010, the entire content of each of which is incorporated by reference herein.

In some embodiments, a droplet comprises an emulsion composition, i.e., a mixture of immiscible fluids (e.g., water and oil). In some embodiments, a droplet is an aqueous droplet that is surrounded by an immiscible carrier fluid (e.g., oil). In some embodiments, a droplet is an oil droplet that is surrounded by an immiscible carrier fluid (e.g., an aqueous solution). In some embodiments, the droplets described herein are relatively stable and have minimal coalescence between two or more droplets. In some embodiments, less than 0.0001%, 0.0005%, 0.001%, 0.005%, 0.01%, 0.05%, 0.1%, 0.5%, 1%, 2%, 3%, 4%, 5%, 6%, 7%, 8%, 9%, or 10% of droplets generated coalesce with other droplets.

In an embodiment, the droplet is formed by flowing an oil phase through an aqueous phase. The oil for the oil phase can be synthetic or naturally occurring. In some embodiments, the oil comprises carbon and/or silicon. In some embodiments, the oil comprises hydrocarbon and/or fluorocarbon. Exemplary oils include, but are not limited to, silicone oil, mineral oil, fluorocarbon oil, vegetable oil, or a combination thereof.

The oil phase can comprise a fluorinated base oil which can additionally be stabilized by combination with a fluorinated surfactant such as a perfluorinated polyether. In some embodiments, the base oil comprises one or more of a HFE 7500, FC-40, FC-43, FC-70, or another common fluorinated oil. In some embodiments, the oil phase comprises an anionic fluorosurfactant. In some embodiments, the anionic fluorosurfactant is Ammonium Krytox (Krytox-AS), the ammonium salt of Krytox FSH, or a morpholino derivative of Krytox FSH. Krytox-AS can be present at a concentration of about 0.1%, 0.2%, 0.3%, 0.4%, 0.5%, 0.6%, 0.7%, 0.8%, 0.9%, 1.0%, 2.0%, 3.0%, or 4.0% (w/w). In some embodiments, the concentration of Krytox-AS is about 1.8%. In some embodiments, the concentration of Krytox-AS is about 1.62%. Morpholino derivative of Krytox FSH may be present at a concentration of about 0.1%, 0.2%, 0.3%, 0.4%, 0.5%, 0.6%, 0.7%, 0.8%, 0.9%, 1.0%, 2.0%, 3.0%, or 4.0% (w/w). In some embodiments, the concentration of morpholino derivative of Krytox FSH is about 1.8%. In some embodiments, the concentration of morpholino derivative of Krytox FSH is about 1.62%.

In some embodiments, the oil phase further comprises an additive for tuning the oil properties, such as vapor pressure, viscosity, or surface tension. Non-limiting examples include perfluorooctanol and 1H,1H,2H,2H-Perfluorodecanol. In some embodiments, 1H,1H,2H,2H-Perfluorodecanol is added to a concentration of about 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 1.25%, 1.50%, 1.75%, 2.0%, 2.25%, 2.5%, 2.'75%, or 3.0% (w/w). In some embodiments, 1H,1H,2H,2H-Perfluorodecanol is added to a concentration of about 0.18% (w/w).

In some embodiments, the droplet is formed by flowing an oil phase through an aqueous solution phase having a DNA template and one or more components (e.g., reagents) that are used to determine the presence or absence of the target. In some embodiments, the one or more components used to determine the presence or absence of the target in the aqueous droplet are soluble and/or miscible in water including, but not limited to, one or more salts, buffering agents, reagents (e.g., a releasing agent such as a restriction endonuclease or a protease, PCR components), surfactants, and/or whatever additional components are necessary for a desired reaction(s) that is intended to occur within a formed droplet. All such additional components can be selected to be compatible with the desired reaction or intended assay.

In some embodiments, assay components (e.g., ligase, a restriction endonuclease, a DNA polymerase, dNTPs, and/or a PCR master mix) can be injected into the partition. The assay components can be injected into the partition in any order or simultaneously.

Methods of injecting fluids into partitions are described in, for example, WO 2012/135259 and U.S. 2012/0132288, each of which is incorporated by reference in its entirety.

In some embodiments, at least 500 partitions (e.g., droplets), at least 1000 partitions, at least 2000 partitions, at least 3000 partitions, at least 4000 partitions, at least 5000 partitions, at least 6000 partitions, at least 7000 partitions, at least 8000 partitions, at least 10,000 partitions, at least 15,000 partitions, at least 20,000 partitions, at least 30,000 partitions, at least 40,000 partitions, at least 50,000 partitions, at least 60,000 partitions, at least 70,000 partitions, at least 80,000 partitions, at least 90,000 partitions, at least 100,000 partitions, at least 200,000 partitions, at least 300,000 partitions, at least 400,000 partitions, at least 500,000 partitions, at least 600,000 partitions, at least 700,000 partitions, at least 800,000 partitions, at least 900,000 partitions, at least 1,000,000 partitions, at least 2,000,000 partitions, at least 3,000,000 partitions, at least 4,000,000 partitions, at least 5,000,000 partitions, at least 10,000,000 partitions, at least 20,000,000 partitions, at least 30,000,000 partitions, at least 40,000,000 partitions, at least 50,000,000 partitions, at least 60,000,000 partitions, at least 70,000,000 partitions, at least 80,000,000 partitions, at least 90,000,000 partitions, at least 100,000,000 partitions, at least 150,000,000 partitions, or at least 200,000,000 partitions are formed.

In some embodiments, the droplets that are generated are substantially uniform in shape and/or size. For example, in some embodiments, the droplets are substantially uniform in average diameter. The term “substantially” or “about” refers to the recited number and any value within 10% of the recited number. In some embodiments, the droplets that are generated have an average diameter of about 0.001 microns, about 0.005 microns, about 0.01 microns, about 0.05 microns, about 0.1 microns, about 0.5 microns, about 1 microns, about 5 microns, about 10 microns, about 20 microns, about 30 microns, about 40 microns, about 50 microns, about 60 microns, about 70 microns, about 80 microns, about 90 microns, about 100 microns, about 150 microns, about 200 microns, about 300 microns, about 400 microns, about 500 microns, about 600 microns, about 700 microns, about 800 microns, about 900 microns, or about 1000 microns. In some embodiments, the droplets that are generated have an average diameter of less than about 1000 microns, less than about 900 microns, less than about 800 microns, less than about 700 microns, less than about 600 microns, less than about 500 microns, less than about 400 microns, less than about 300 microns, less than about 200 microns, less than about 100 microns, less than about 50 microns, or less than about 25 microns. In some embodiments, the droplets that are generated are non-uniform in shape and/or size.

In some embodiments, the droplets that are generated are substantially uniform in volume. For example, in some embodiments, the droplets that are generated have an average volume of about 0.001 nL, about 0.005 nL, about 0.01 nL, about 0.02 nL, about 0.03 nL, about 0.04 nL, about 0.05 nL, about 0.06 nL, about 0.07 nL, about 0.08 nL, about 0.09 nL, about 0.1 nL, about 0.2 nL, about 0.3 nL, about 0.4 nL, about 0.5 nL, about 0.6 nL, about 0.7 nL, about 0.8 nL, about 0.9 nL, about 1 nL, about 1.5 nL, about 2 nL, about 2.5 nL, about 3 nL, about 3.5 nL, about 4 nL, about 4.5 nL, about 5 nL, about 5.5 nL, about 6 nL, about 6.5 nL, about 7 nL, about 7.5 nL, about 8 nL, about 8.5 nL, about 9 nL, about 9.5 nL, about 10 nL, about 11 nL, about 12 nL, about 13 nL, about 14 nL, about 15 nL, about 16 nL, about 17 nL, about 18 nL, about 19 nL, about 20 nL, about 25 nL, about 30 nL, about 35 nL, about 40 nL, about 45 nL, or about 50 nL.

In some embodiments, the partitions (e.g., droplets) are stable and are capable of long-term storage. In some embodiments, the partitions are stored at about −70°, −20°, 0°, 3°, 4°, 5°, 6°, 7°, 8°, 9°, 10°, 15°, 20°, 25°, 30°, 35°, or 40° C. for an extended period of time (e.g., for at least 30 days, at least 60 days, at least 90 days, or longer).

Partitions as described herein can contain one or more surfactants to reduce coalescence of droplets during transport. As used herein, a “surfactant” is a surface-active substance capable of reducing the surface tension of a liquid in which it is present. A surfactant, which also or alternatively is described as a detergent and/or a wetting agent, can incorporate both a hydrophilic portion and a hydrophobic portion, which can collectively confer a dual hydrophilic-hydrophobic character on the surfactant. A surfactant can, in some cases, be characterized according to its hydrophilicity relative to its hydrophobicity. In some embodiments, the aqueous phase incorporates at least one hydrophilic surfactant. The aqueous phase can include at least one nonionic surfactant and/or ionic surfactant. In certain embodiments, the aqueous phase includes a surfactant that is a block copolymer of polypropylene oxide and polyethylene oxide. In some embodiments, the surfactant is a block copolymer of polypropylene oxide and polyethylene oxide sold under the trade names PLURONIC and TETRONIC (BASF). In some embodiments, the surfactant is a nonionic block copolymer of polypropylene oxide and polyethylene oxide sold under the trade name PLURONIC F-68. In some embodiments, the surfactant of the aqueous phase is a water-soluble and/or hydrophilic fluorosurfactant. Exemplary fluorosurfactants for the aqueous phase are sold under the trade name ZONYL (DuPont), such as ZONYL FSN fluorosurfactants. In some cases, the surfactant can include polysorbate 20 (sold under the trade name TWEEN-20 by ICI Americas, Inc.). The concentration of a particular surfactant or total surfactant present in the aqueous phase can be selected to stabilize emulsion droplets prior to heating. In some embodiments, the concentration of surfactant for the aqueous phase is 0.01 to 10%, 0.05 to 5%, 0.1 to 1%, or 0.5% by weight.

In exemplary step 190, the DNA template is amplified in the subset of partitions. The DNA template can be amplified by, for example, PCR, LCR (Ligase Chain Reaction), SDA (Strand Displacement Amplification), 3 SR (Self-Sustained Synthetic Reaction), TMA (Transcription-Mediated Amplification), rolling circle amplification (RCA), or hyper-branched RCA (HRCA).

In exemplary step 200, the presence or absence of the target is detected in the sample by detecting the presence or absence of the amplified DNA template in the plurality of partitions. In some embodiments, the amplified DNA template is detected by direct incorporation of a label (e.g., a fluorophore, a radioisotope, or an enzyme) into the amplified DNA template by using label-conjugated primers or nucleotides. In some embodiments, a dye that fluoresces when it intercalates into double-stranded DNA is used to detect the amplified DNA template. Exemplary intercalating dyes include, but are not limited to, ethidium bromide, propidium iodide, and SYBR™ green. In some embodiments, the amplified DNA template is detected by using a DNA probe having a reporter on one end and a quencher on the other end. In some embodiments, the probe comprises a reporter-quencher combination as employed in a double-stranded probe, a TAQMAN™ probe, a molecular beacon probe, a SCORPION™ probe, a dual hybridization probe, or an ECLIPSE™ probe.

The detectable label (e.g., a label as described herein) can be detected using any of a variety of detector devices. Exemplary detection methods include optical absorbance detection (e.g., fluorescence or chemiluminescence) or radioactive detection. As a non-limiting example, a fluorescent label can be detected using a detector device equipped with a module to generate excitation light that can be absorbed by a fluorophore, as well as a module to detect light emitted by the fluorophore.

In some embodiments, the detector further comprises handling capabilities for the partitioned samples (e.g., droplets), with individual partitioned samples entering the detector, undergoing detection, and then exiting the detector. In some embodiments, partitioned samples (e.g., droplets) are detected serially while the partitioned samples are flowing. In some embodiments, partitioned samples (e.g., droplets) are arrayed on a surface and a detector moves relative to the surface, detecting signal(s) at each position containing a single partition. Examples of detectors are provided in WO 2010/036352, the contents of which are incorporated herein by reference. In some embodiments, detectable labels in partitioned samples are detected serially without flowing the partitioned samples (e.g., using a chamber slide).

Following acquisition of fluorescence detection data, a general purpose computer system (referred to herein as a “host computer”) can be used to store and process the data. Computer-executable logic can be employed to perform such functions as subtraction of background signal, assignment of target and/or reference sequences, and qualification and/or quantification of the data. A host computer can be useful for displaying, storing, retrieving, or calculating diagnostic results from the molecular profiling; storing, retrieving, or calculating raw data from expression analysis; or displaying, storing, retrieving, or calculating any sample or patient information useful in the methods of the present invention.

The host computer can be configured with many different hardware components and can be made in many dimensions and styles (e.g., desktop PC, laptop, tablet PC, handheld computer, server, workstation, mainframe). Standard components, such as monitors, keyboards, disk drives, CD and/or DVD drives, and the like, can be included. Where the host computer is attached to a network, the connections can be provided via any suitable transport media (e.g., wired, optical, and/or wireless media) and any suitable communication protocol (e.g., TCP/IP); the host dcomputer can include suitable networking hardware (e.g., modem, Ethernet card, WiFi card). The host computer can implement any of a variety of operating systems, including UNIX, Linux, Microsoft Windows, MacOS, or any other operating system.

Computer code for implementing aspects of the present invention can be written in a variety of languages, including PERL, C, C++, Java, JavaScript, VBScript, AWK, or any other scripting or programming language that can be executed on the host computer or that can be compiled to execute on the host computer. Code can also be written or distributed in low level languages such as assembler languages or machine languages.

The host computer system advantageously provides an interface via which the user controls operation of the tools. In the examples described herein, software tools are implemented as scripts (e.g., using PERL), execution of which can be initiated by a user from a standard command line interface of an operating system such as Linux or UNIX. Those skilled in the art will appreciate that commands can be adapted to the operating system as appropriate. In other embodiments, a graphical user interface can be provided, allowing the user to control operations using a pointing device. Thus, the present invention is not limited to any particular user interface.

Scripts or programs incorporating various features of the invention as described herein can be encoded on various computer readable media for storage and/or transmission. Examples of suitable media include magnetic disk or tape, optical storage media such as compact disk (CD) or DVD (digital versatile disk), flash memory, and carrier signals adapted for transmission via wired, optical, and/or wireless networks conforming to a variety of protocols, including the Internet.

In some embodiments, the method 100 further comprises quantifying the target by determining a number of partitions comprising the target and a total number of partitions. Once a binary “yes-no” result has been determined for each of the partitions, the data for the partitions is analyzed by an algorithm based on Poisson statistics to quantify the amount of target in the sample. Statistical method for quantifying the concentration or amount of target or targets is described, for example in the aforementioned WO 2010036352.

### KITS

In another aspect, kits for detecting the presence or absence of a target in a sample according to the methods described herein are provided. In some embodiments, a kit comprises a first affinity agent linked to a solid support, a second affinity agent conjugated to a first oligonucleotide, and a third affinity agent conjugated to a second oligonucleotide, all of which are described herein. In some embodiments, the kit further comprises assay components (e.g., a ligase, a restriction endonuclease, a DNA polymerase, dNTPs, a buffer, a PCR master mix). In some embodiments, the kit further comprises instructions for carrying out the methods described herein.

### EXAMPLES

The following examples are provided by way of illustration only and not by way of limitation. Those of skill in the art will readily recognize a variety of non-critical parameters that could be changed or modified to yield essentially the same or similar results.

### Example 1

### Quantitation of Target with a Digital Proximity Assay

This example illustrates the feasibility of quantitating a target with a digital proximity assay according to the invention. The example includes a protocol for an assay with a known concentration of protein target, followed by analysis that correlates the measured concentration with the known protein concentration, thus demonstrating absolute target quantitation.

Recombinant purified Human Prostate Specific Antigen (PSA) is from R&D systems. Three monoclonal PSA antibodies (against three different epitopes) are from Meridian Life Science. Two of the monoclonal antibodies are biotinylated using EZ-Link Sulfo-NHS-LC-Biotin (Thermo Scientific) according to the manufacturer's protocol. PLA probes are prepared with the biotinylated antibodies using TaqMan Protein Assay Oligo Probe Kit (Thermo Scientific) according to the manufacturer's protocol.

The third antibody, serves as the capture, is bound to Carboxylic acid modified magnetic beads (Thermo Scientific) according to the manufacturer's protocol, such that the final antibody coating density is 1.5 μg antibody per mg beads.

A dilution series of purified PSA protein is prepared in 5-fold increments ranging from 1 nM to 1 fM, inside PLA buffer (1×PBS pH 7.4, 1 mM D-Biotin (Life Technologies), 1 mg per ml BSA (New England Biolabs), 0.05% Tween-20 (Sigma-Aldrich), 100 nM goat IgG (Sigma-Aldrich), 0.1 mg/ml salmon sperm DNA (Life Technologies), 5 mM EDTA). Each binding reaction includes 45 μl of serially diluted PSA and 1 μl of 5 mg/ml antibody-coated magnetic beads (equivalent to 50 fmole antibody per reaction), and incubated for 1 hour at 37° C. on a rotator at 20 rpm, to enable binding of the antigen to the antibodies. Following incubation, unbound PSA is removed by magnetizing the beads to the tube walls and carefully pipetting the supernatant. Beads are then washed with 500 μl of wash buffer (1xPBS pH 7.4 with 0.05% (vol/vol) Tween-20), twice, and subsequently the wash buffer is removed.

To each reaction tube, 50 μl of probes mix (1 nM of each of the probes in PLA buffer or 50 fmole probe per reaction) is added and the reaction is incubated 1 hour at 37° C. on a rotator at 20 rpm. Unbound probes are removed by magnetizing the beads to the tubes walls and carefully pipetting the supernatant. Beads are then washed with 100 μl of wash buffer twice, and subsequently the wash buffer is removed.

To each reaction tube, 50 μl of ligation solution (Thermo's TaqMan Protein Assay kit, prepared according to the manufacturer's protocol) is added and the reaction is incubated for 10 minutes at 37° C. To each reaction tube, 2 microliter of diluted protease (Thermo's TaqMan Protein Assay kit, prepared according to the manufacturer's protocol) is added and the reaction is incubated for 10 minutes at 37° C. followed by 5 minutes at 95° C. For the ligated template recovery, beads are magnetized and supernatant is transferred to a new tube.

Detection is performed by using droplet digital PCR as follows: 11 μl of each ligation template are mixed with 1μl of the Universal PCR Assay 20× (Thermo's TaqMan Protein Assay kit) and 12 μl of ddPCR supermix for probes (Bio-Rad). Droplets are generated using QX200™ Droplet Generator, cycled in a C1000 Thermal Cycler, and read using QX200™ Droplet Reader, all from Bio-Rad, according to the manufacturer's protocols.

For analysis, the theoretical concentration of ligation template inside the droplets is calculated from the original target concentration in the sample, assuming a single template molecule is generated from each target molecule. Then, a graph is plotted correlating the results measured by the ddPCR (template copies per microliter) against the theoretical ligation template concentration inside the droplets for each sample. The dynamic range of the assay is the concentration range at which a one to one ratio is observed between the theoretical and measured target concentrations.

All patents, patent applications, and other published reference materials cited in this specification are hereby incorporated herein by reference in their entirety.

What is claimed is:

1. A method of determining the presence or absence of a target in a sample,
the method comprising:
contacting the sample with a first affinity agent linked to a solid support, wherein the first affinity agent specifically binds to a first epitope on the target;
washing the solid support;
incubating the solid support with a solution having a second affinity agent capable of specifically binding to a second epitope on the target and a third affinity agent capable of specifically binding to a third epitope on the target, wherein the second affinity agent is attached to a first oligonucleotide and the third affinity agent is attached to a second oligonucleotide capable of interacting directly or indirectly with the first oligonucleotide when the first and second oligonucleotides are in close proximity;
allowing the first and second oligonucleotides to interact by
(a) hybridization of a third oligonucleotide complementary to at least a portion of each of the first and second oligonucleotides followed by ligation of the first oligonucleotide to the second oligonucleotide; or
(b) hybridization of the first oligonucleotide to the second oligonucleotide;
washing the solid support;
optionally, forming a DNA template;
releasing the DNA template and forming a solution comprising the DNA template;
forming a plurality of partitions from the solution, wherein a subset of the partitions contains the DNA template;
amplifying the DNA template in the subset of partitions; and
detecting the presence or absence of the target in the sample by detecting the presence or absence of the amplified DNA template in the plurality of partitions.

2. The method of claim 1, wherein the first, second and third epitopes are at least partially overlapping.

3. The method of claim 1, wherein the second epitope is located on a first target and the third epitope is located on a second target.

4. The method of claim 1, wherein the target has a repeating identical epitope such that the first and second affinity agents or the first and third affinity agents recognize the same epitope.

5. The method of claim 4, wherein the target is a dimeric protein or an aggregate-forming protein.

6. The method of claim 1, further quantifying the target by determining a number of partitions comprising the target and a total number of partitions.

7. The method of claim 1, wherein each of the plurality of partitions is a droplet.

8. The method of claim 1, wherein the forming the DNA template step comprises extending the 3′ terminus of the third oligonucleotide.

9. The method of claim 1, wherein the forming the DNA template step comprises extending the 3′ terminus of the first oligonucleotide and/or the second oligonucleotide.

10. The method of claim 1, wherein the hybridization of the first oligonucleotide to the second oligonucleotide comprises forming the DNA template step.

11. The method of claim 1, wherein the concentration of the second and third affinity agents is at least equal to the concentration of the first affinity agent.

12. The method of claim 1, wherein the releasing step comprises cleaving a site in the DNA template by a restriction endonuclease.

13. The method of claim 1, wherein the releasing step comprises physical, chemical or enzymatic cleavage of the DNA template.

14. The method of claim 1, wherein the releasing step comprises cleaving at least a portion of an affinity agent-oligonucleotide complex by a protease or by a change in pH.

15. The method of claim 1, wherein the target is a protein or a protein aggregate.

16. The method of claim 1, wherein the first, second, and third affinity agents are each an antibody or an antibody fragment.

17. The method of claim 1, wherein the amplifying step comprises PCR.

18. The method of claim 1, wherein the solid support is a particle or a surface of a reaction vessel.

19. A kit for detecting the presence or absence of a target in a sample, the kit comprising:
a first affinity agent linked to a solid support, wherein the first affinity agent specifically binds to a first epitope on the target; and
a second affinity agent capable of specifically binding to a second epitope on the target and a third affinity agent capable of specifically binding to a third epitope on the target, wherein the second affinity agent is conjugated to a first oligonucleotide and the third affinity agent is conjugated to a second oligonucleotide capable of interacting directly or indirectly with the first oligonucleotide when the first and second oligonucleotides are in close proximity.

20. The kit of claim 19, further comprising at least one component consisting of a ligase, a restriction endonuclease, a DNA polymerase, dNTPs, a buffer, a PCR master mix, and instructions for performing a method of detecting a target in a sample.
